(19) United States
(12) Patent Application Publication
(10) Pub. No.: US 2008/0145455 A1
(43) Pub. Date: Jun. 19, 2008

(54) Combination of Inorganic Hemostatic Agents with Other Hemostatic Agents

(76) Inventor: Robert L. Bedard, McHenry, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/610,426

(22) Filed: Dec. 13, 2006

(51) Int. Cl. A61K 33/06

(52) U.S. Cl. 424/682

(57) ABSTRACT

Several hemostatic bandages that are commercially available function by tightly sealing a wound so that hemostasis can occur before excessive blood loss takes place. Hemostatic bandages which seal a wound but which do not actually speed the coagulation mechanism include chitosan and chitin-based bandages. Hemostatic gauzes based on chemically modified cellulose are also commercially available. These bandages and gauzes, used in combination with various inorganic materials such as zeolite, silica, and/or diatomaceous earth powder show a synergistic effect. It has been found that the inorganic material activates the contact coagulation mechanism while the biopolymer component binds the wound and prevents excessive blood loss.

### FIELD OF THE INVENTION

The present invention relates to blood clotting agents/medical devices and methods of controlling bleeding in animals and humans. More particularly, the present invention relates to the effectiveness of a number of different inorganic materials in combination with biomaterials comprising chitosan and/or other hydrophilic polymers, (labeled COMBINATION MATERIAL, herein) to control bleeding, including severe bleeding. The material may contain polyacrylic acid with or without other polymers. The blood clotting agents/medical devices of the present invention function to substantially stanch the flow of blood from a wound by both accelerating the clotting process and adhering to the wound site, sealing the wound, accelerating blood clot formation at the wound site, reinforcing clot formation at the wound site, and preventing blood flow from the wound site.

### BACKGROUND OF THE INVENTION

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some instances, the wound and the bleeding are minor, and normal blood clotting functions without significant outside aid in stopping the bleeding. Unfortunately, in other circumstances, substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner. Moreover, severe wounds can be inflicted in very remote areas or in situations, such as on a battlefield, where adequate medical assistance is not immediately available. In these instances, it is important to stop bleeding, even in less severe wounds, long enough to allow the injured person or animal to receive medical attention. In addition, it may be desirable to accelerate the clotting of even minor wounds to allow the injured person to resume their normal activities.

In an effort to address the above-described problems, materials have been developed for controlling excessive bleeding in situations where conventional aid is unavailable or less than optimally effective. Although these materials have been shown to be somewhat successful, they are not effective enough for traumatic wounds and tend to be expensive. Furthermore, these materials are sometimes ineffective in all situations and can be difficult to apply as well as remove from a wound. Additionally, or alternatively, some materials, especially those of organic origin, can produce undesirable side effects.

Compositions for promoting the formation of clots in blood have also been developed. Such compositions include those that contain zeolites and binders. The use of activated zeolites was disclosed by Hursey et al. in U.S. Pat. No. 4,822,349. It was recognized that the use of these activated zeolites in the clotting of blood generated heat and Hursey et al. stated that the heat was important in achieving a cauterization effect as well as increasing coagulation of the blood. In US 2005/0074505 A1, there is described the use of a zeolite that is exchanged with calcium ions to a very high level. Currently clay-bound Ca-exchanged zeolite A is being sold in an activated form by Z-Medica as a hemostatic treatment for hemorrhages. On some occasions, this calcium exchanged zeolite A has been reported to exhibit an undesirable exothermic effect upon use.

Currently available hemostatic bandages such as collagen wound dressings or dry fibrin thrombin wound dressings are restricted to use in surgical applications, and are not sufficiently resistant to dissolution in high blood flow. They also do not possess enough hemostatic properties to serve any practical purpose in the stanching of severe blood flow. These currently available surgical hemostatic bandages are also delicate and thus prone to failure should they be damaged by bending or loading with pressure. They are also susceptible to dissolution in hemorrhagic bleeding. Such dissolution and collapse of these bandages may be catastrophic, because it can produce a loss of adhesion to the wound and allow bleeding to continue unabated. In a recent patent application published by Campbell et al, US 2005/0137512, which is incorporated herein in its entirety, is disclosed a wound dressing comprising hydrophilic polymers such as chitosan that functions to seal a wound and substantially stanch bleeding. While this wound dressing functions to seal a wound, in testing such materials in connection with the present invention, it has been found that these wound dressings fail to enhance the clotting of blood.

Blood clot formation is a complex process. Several principles are useful in understanding coagulation. In general, the clotting proteins circulate normally as inactive precursors. Coagulation involves a series of activation reactions that in turn act as the catalysts for the next level of reactions and hence, the frequent term “coagulation cascade”. During the reaction(s) process, these proteins and the fibrin mass itself, is highly unstable and water-soluble. This unstable condition will continue until the very final aspects of coagulation. In addition, without (or in limited quantities) those clotting proteins (or in the presence of anticoagulants, i.e., heparin), clotting becomes delayed or prolonged. Eventually, however, fibrin (the foundation of a blood clot) will be formed. This occurs with the cleaving of fibrinogen, one of the coagulation proteins. Finally, Factor XIII (stabilizing factor) is activated by thrombin to yield cross-linked fibrin, which is highly insoluble and stable in formation. In certain situations it is highly desirable to accelerate this process as is disclosed in the present invention.

### SUMMARY OF THE INVENTION

The invention is directed to a first-aid/primary intervention wound treatment for control of bleeding, especially severe, life-threatening bleeding. There is a need for low cost wound dressings that are suitable for control of severe life-threatening bleeding as well as less severe bleeding. There is a need for this type of dressing especially in the battlefield, where typically 50% of all deaths are associated with an inability to immediately control severe bleeding. The present invention is capable of substantially stanching the flow of life-threatening bleeding from a wound by adhering to the wound site, sealing the wound, greatly accelerating blood clot formation at the wound site, reinforcing clot formation at the wound site, preventing bleed out from the wound site, and substantially prohibiting the flow of blood out of the wound site. It has been found that a combination of a hydrophilic polymer, such as chitosan together with an inorganic powder such as a zeolite, diatomaceous earth or silica powder provides an improved hemostatic device. Other effective inorganic powders may be used as well. The hydrophilic polymer may include alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be of amylase, amylopectin and a combination of amylopectin and amylase. Preferably, the hydrophilic polymer is chitosan. Preferably, the chitosan has a weight average molecular weight of at least about 100 kDa. More preferably, the chitosan has a weight average molecular weight of at least about 150 kDa. Most preferably, the chitosan has a weight average molecular weight of at least about 300 kDa.

The medical device may further comprise an active ingredient. The active ingredient may include, but is not limited to, calcium, thrombin, factor VIIa, factor XIII, thromboxane A2, prostaglandin-2a, epidermal growth factor, platelet derived growth factor, Von Willebrand factor, tumor necrosis factor (TNF), TNF-alpha, transforming growth factor (TGF), TGF-alpha, TGF-beta, insulin like growth factor, fibroblast growth factor, keratinocyte growth factor, nerve growth factor, penicillin, ampicillin, methicillin, amoxycillin, clavamox, clavulanic acid, amoxicillin, aztreonam, imipenem, streptomycin, Kanamycin, Tobramycin, gentamicin, vancomycin, clindamycin, erythromycin, polymyxin, bacitracin, amphotericin, nystatin, rifampicin, tetracycline, doxycycline, chloramphenicol and combinations thereof.

In another embodiment, a compressed composite sponge for hemorrhage control comprising a hydrophilic polymer sponge and a wettable polymer matrix or wettable polymer matrices inside the sponge and/or at the sponge surface is provided together with an inorganic powder such as a zeolite, diatomaceous earth or silica powder. The hydrophilic polymer may include alginate, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be amylase, amylopectin and a combination of both amylopectin and amylase.

The wettable polymer may include non-woven mats, woven mats, molded polymer mesh and low density sponges. The wettable polymer may include, but is not limited to a chitin, an alginate, a neutralized chitosan, a re-acetylated chitosan, a poly(glycolic acid), a poly(lactic acid), a poly(e-caprolactone), a poly(.beta.-hydroxybutyric acid), a poly(.beta.-hydroxyvaleric acid), a polydioxanone, a poly(ethylene oxide), a poly(malic acid), a poly(tartronic acid), a polyphosphazene, a polyethylene, a polypropylene, a metallocene polymer, a polyurethane, a polyvinylchloride polymer, a polyester, a polyamide and combinations thereof. Preferably, the hydrophilic polymer is chitosan.

It has been found that many inorganic materials will accelerate the coagulation of blood. In particular, it has been found that solids that can be used to activate the coagulation of platelet-poor plasma in the APTT clinical test or whole blood in the ACT clinical test will also serve as a coagulation accelerator in vivo. In addition, a variety of other materials have been found that can also accelerate blood clotting. Typical materials that can be used for in-vivo clotting include diatomaceous earth, glass powder or fibers, precipitated or fumed silica, kaolin and montmorillonite clays, Ca exchanged permutites. These materials can be used in an aqueous slurry, dry powder or dehydrated forms, and can also be bound with suitable organic or inorganic binders and/or contained in sheet or bandage form.

### DETAILED DESCRIPTION OF THE INVENTION

The invention is directed to a first-aid/primary intervention wound treatment for control of bleeding, especially severe, life-threatening bleeding. There is a need for low cost wound dressings that are suitable for control of severe life-threatening bleeding as well as less severe bleeding. There is a need for this type of dressing especially in the battlefield, where typically 50% of all deaths are associated with an inability to immediately control severe bleeding. The present invention is capable of substantially stanching the flow of life-threatening bleeding from a wound by adhering to the wound site, sealing the wound, greatly accelerating blood clot formation at the wound site, reinforcing clot formation at the wound site, preventing bleed out from the wound site, and substantially prohibiting the flow of blood out of the wound site. It has been found that a combination of a hydrophilic polymer, such as chitosan together with an inorganic powder such as a zeolite, diatomaceous earth or silica powder provides an improved hemostatic device. Other effective inorganic powders may be used as well. The hydrophilic polymer may include alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be of amylase, amylopectin and a combination of amylopectin and amylase. Preferably, the hydrophilic polymer is chitosan. Preferably, the chitosan has a weight average molecular weight of at least about 100 kDa. More preferably, the chitosan has a weight average molecular weight of at least about 150 kDa. Most preferably, the chitosan has a weight average molecular weight of at least about 300 kDa.

The medical device may further comprise an active ingredient. The active ingredient may include, but is not limited to, calcium, thrombin, factor VIIa, factor XIII, thromboxane A2, prostaglandin-2a, epidermal growth factor, platelet derived growth factor, Von Willebrand factor, tumor necrosis factor (TNF), TNF-alpha, transforming growth factor (TGF), TGF-alpha, TGF-beta, insulin like growth factor, fibroblast growth factor, keratinocyte growth factor, nerve growth factor, penicillin, ampicillin, methicillin, amoxycillin, clavamox, clavulanic acid, amoxicillin, aztreonam, imipenem, streptomycin, Kanamycin, Tobramycin, gentamicin, vancomycin, clindamycin, erythromycin, polymyxin, bacitracin, amphotericin, nystatin, rifampicin, tetracycline, doxycycline, chloramphenicol and combinations thereof.

In another embodiment, a compressed composite sponge for hemorrhage control comprising a hydrophilic polymer sponge and a wettable polymer matrix or wettable polymer matrices inside the sponge and/or at the sponge surface is provided together with an inorganic powder such as a zeolite, diatomaceous earth or silica powder. The hydrophilic polymer may include alginate, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be amylase, amylopectin and a combination of both amylopectin and amylase.

The wettable polymer may include non-woven mats, woven mats, molded polymer mesh and low density sponges. The wettable polymer may include, but is not limited to a chitin, an alginate, a neutralized chitosan, a re-acetylated chitosan, a poly(glycolic acid), a poly(lactic acid), a poly(e-caprolactone), a poly(.beta.-hydroxybutyric acid), a poly(.beta.-hydroxyvaleric acid), a polydioxanone, a poly(ethylene oxide), a poly(malic acid), a poly(tartronic acid), a polyphosphazene, a polyethylene, a polypropylene, a metallocene polymer, a polyurethane, a polyvinylchloride polymer, a polyester, a polyamide and combinations thereof. Preferably, the hydrophilic polymer is chitosan.

It has been found that many inorganic materials will accelerate the coagulation of blood. Typical materials that can be used for in-vivo clotting include zeolites, diatomaceous earth, glass powder or fibers, precipitated or fumed silica, kaolin and montmorillonite clays, Ca exchanged permutites. These materials can be used in an aqueous slurry, dry powder or dehydrated forms, and can also be bound with suitable organic or inorganic binders and/or contained in sheet or bandage form.

Diatomaceous earth is a naturally occurring, soft, chalk-like sedimentary rock that is easily crumbled into a fine white to off-white powder. This powder has an abrasive feel, similar to pumice powder and is very light, due to its high porosity. It is composed primarily of silica and consists of fossilized remains of diatoms, a type of hard-shelled algae.

Bioactive glasses are a group of surface reactive glass-ceramics and include the original bioactive glass, Bioglass®. The biocompatibility of these glasses has led them to be investigated extensively for use as implant materials in the human body to repair and replace diseased or damaged bone.

The apparatus that was used was a TEG® analyzer from Haemoscope Corp. of Morton Grove, Ill. This apparatus measures the time until initial fibrin formation, the kinetics of the initial fibrin clot to reach maximum strength and the ultimate strength and stability of the fibrin clot and therefore its ability to do the work of hemostasis—to mechanically impede hemorrhage without permitting inappropriate thrombosis.

On unactivated samples:
i. Pipet 360 uL from red topped tube into cup, start TEG test
   On activated samples:
i. First, obtain the sample to be tested from lab. They should be weighed, bottled, oven activated (if needed), and capped prior to the start of the experiment. COMBINATION MATERIAL samples are bottled in twice the amount that needs to be tested. For example, if channel two is to test 5 mg of COMBINATION MATERIAL A and blood, the amount weighed out in the bottle for channel two will be 10 mg. For 10 mg samples, 20 mg is weighed out, etc. See note below for reason.
ii. For one activated run, 3 COMBINATION MATERIAL samples were tested at a time. An unactivated blood sample with no additive is run in the first channel. Channels 2, 3 and 4 are blood samples contacted with a COMBINATION MATERIAL.
iii. Once ready to test, set one pipet to 720 uL and other pipet to 360 uL. Prepare three red capped tubes (plain polypropylene-lined tubes without added chemicals) to draw blood and prepare three red additional capped tubes to pour the COMBINATION MATERIAL sample into.
iv. Draw blood from volunteer and bring back to TEG analyzer. Discard the first tube collected to minimize tissue factor contamination of blood samples. Blood samples were contacted with COMBINATION MATERIAL and running in TEG machine prior to an elapsed time of 4-5 minutes from donor collection.
v. Open bottle 1 and pour COMBINATION MATERIAL into red capped tube.
vi. Immediately add 720 uL of blood to COMBINATION MATERIAL in tube.
vii. Invert 5 times.
viii. Pipet 360 uL of blood and COMBINATION MATERIAL mixture into cup.
ix. Start TEG test.

Note: The proportions are doubled for the initial mixing of blood and COMBINATION MATERIAL because some volume of blood is lost to the sides of the vials, and some samples absorb blood. Using double the volume ensures that there is at least 360 uL of blood to pipet into cup. The proportion of COMBINATION MATERIAL to blood that we are looking at is usually 5 mg/360 uL, 10 mg/360 uL, and 30 mg/360 uL

The R(min) reported in the Tables below is the time from the start of the experiment to the initial formation of the blood clot as reported by the TEG analyzer. The TEG® analyzer has a sample cup that oscillates back and forth constantly at a set speed through an arc of 4°45′. Each rotation lasts ten seconds. A whole blood sample of 360 ul is placed into the cup, and a stationary pin attached to a torsion wire is immersed into the blood. When the first fibrin forms, it begins to bind the cup and pin, causing the pin to oscillate in phase with the clot. The acceleration of the movement of the pin is a function of the kinetics of clot development. The torque of the rotating cup is transmitted to the immersed pin only after fibrin-platelet bonding has linked the cup and pin together. The strength of these fibrin-platelet bonds affects the magnitude of the pin motion, such that strong clots move the pin directly in phase with the cup motion. Thus, the magnitude of the output is directly related to the strength of the formed clot. As the clot retracts or lyses, these bonds are broken and the transfer of cup motion is diminished. The rotation movement of the pin is converted by a mechanical-electrical transducer to an electrical signal which can be monitored by a computer.

The resulting hemostasis profile is a measure of the time it takes for the first fibrin strand to be formed, the kinetics of clot formation, the strength of the clot (in shear elasticity units of dyn/cm²) and dissolution of clot. The following data has been collected from volunteer donors. In each case, the unadulterated blood data is included with the data after addition of known amounts of materials.

TABLE 1
Chitosan Bandage

| Bandage, Run, Amount | R(min) |
|---|---|
| Run 4 - Hemcon bandage 5 mg | 26.4 |
| Run 4 - Hemcon bandage 10 mg | 20.9 |
| Run 4 - Hemcon bandage 30 mg | 23.5 |
| Run 9 - Hemcon bandage 5 mg | 24.4 |
| Run 9 - Hemcon bandage 10 mg | 21.2 |
| Control - Run 4 | 25.2 |
| Control - Run 9 | 19.3 |

TABLE 2
Chitosan and Inorganic hemostatic agent

| Bandage, Run, Amount | R(min) |
|---|---|
| Run 15 - Hemcon 5 mg, Ca4A zeolite 5 mg | 3.4 |
| Run 15 - Hemcon 10 mg, Ca4A zeolite 5 mg | 3.5 |
| Run 15 - Hemcon 30 mg, Ca4A zeolite 5 mg | 2.0 |
| Run 7 - Hemcon 5 mg, Ca4A zeolite 5 mg | 3.5 |
| Run 7 - Hemcon 10 mg, Ca4A zeolite 5 mg | 3.7 |
| Run 7 - Hemcon 30 mg, Ca4A zeolite 5 mg | 2.9 |
| Run 5 - Hemcon 5 mg, Celite 270, 5 mg | 1.5 |
| Run 5 - Hemcon 10 mg, Celite 270, 5 mg | 1.5 |
| Run 5 - Hemcon 30 mg, Celite 270, 5 mg | 2.2 |
| Run 1 - Hemcon 5 mg, Celite 270, 5 mg | 1.8 |
| Run 1 - Hemcon 10 mg, Celite 270, 5 mg | 1.8 |
| Run 1 - Hemcon 30 mg, Celite 270, 5 mg | 0.9 |
| Run 9 - Hemcon 5 mg, HI-SIL250, 5 mg | 2.0 |
| Run 9 - Hemcon 10 mg, HI-SIL250, 5 mg | 2.2 |
| Run 9 - Hemcon 30 mg, HI-SIL250, 5 mg | 2.4 |
| Run 3 - Hemcon 5 mg, HI-SIL250, 5 mg | 1.9 |
| Run 3 - Hemcon 10 mg, HI-SIL250, 5 mg | 1.8 |
| Run 3 - Hemcon 30 mg, HI-SIL250, 5 mg | −61.4 |
| Run 15 control | 31.2 |
| Run 7 control | 30.9 |
| Run 5 control | 34.1 |
| Run 1 control | 24.2 |
| Run 9 control | 24.3 |
| Run 3 control | 20.8 |

The materials studied include the following:
1. The Hemcon bandage is a 2 inch by 2 inch shrimp shell-derived chitosan pad obtained from Hemcon Medical Technologies Inc. in Portland Oreg.
2. Ca4A zeolite is obtained by calcium ion exchange of NaA zeolite, a micron-sized powder obtained from UOP LLC, Des Plaines Ill.
3. Celite 270 is a low surface area 4-6 m²/g diatomaceous earth obtained from World Minerals Inc., headquartered in Santa Barbara, Calif., USA
4. Hi-Sil 250 is a precipitated silica (silica gel) obtained from PPG Industries, Pittsburgh, Pa.

Highly significant clot acceleration was observed with the combinations of the Hemcon chitosan containing bandage and each of the calcium exchanged zeolite, the diatomaceous earth and the silica gel. The bandage when used alone did not accelerate clotting and in some instances resulted in delayed clotting. In light of this negative effect from the chitosan containing bandage it was surprising that the mixtures of the present invention were very effective in accelerating the hemostatic process.

Other appropriate hemostatic or absorptive agents may also be added. These include but are not limited to chitosan and its derivatives, fibrinogen and its derivatives (represented herein as fibrin(ogen), e.g. fibrin, which is a cleavage product of fibrinogen, or super-absorbent polymers of many types, cellulose of many types, other cations such as calcium, silver, and sodium or anions, other ion exchange resins, and other synthetic or natural absorbent entities such as super-absorbent polymers with and without ionic or charge properties.

In addition, the COMBINATION MATERIAL may in addition have added to it vasoactive or other agents which promote vasoconstriction and hemostasis. Such agents might include catecholamines or vasoactive peptides. This may be especially helpful in its dry form so that when blood is absorbed, the additive agents become activated and are leached into the tissues to exert their effects. In addition, antibiotics and other agents which prevent infection (any bacteriocidal or bacteriostatic agent or compound) and anesthetics/analgesics may be added to enhance healing by preventing infection and reducing pain. In addition, fluorescent agents or components could be added to help during surgical removal of some forms of the mineral to ensure minimal retention of the mineral after definitive control of hemorrhage is obtained.

The formulations of the present invention may be administered to a site of bleeding by any of a variety of means that are well known to those of skill in the art. Examples include but are not limited to internally (e.g. by ingestion of a liquid or tablet form), directly to a wound, (e.g. by shaking powdered or granulated forms of the material directly into or onto a site of hemorrhage), by placing a material such as a bandage that is impregnated with the material into or onto a wound, by spraying it into or onto the wound, or otherwise coating the wound with the material. Bandages may also be of a type that, with application of pressure, bend and so conform to the shape of the wound site. Partially hydrated forms resembling mortar or other semisolid-semiliquid forms, etc. may be used to fill certain types of wounds. For intra-abdominal bleeding, we envision puncture of the peritoneum with a trocar followed by administration of the COMBINATION MATERIAL of various suitable formulations.

Formulations may thus be in many forms such as bandages of varying shapes, sizes and degrees of flexibility and/or rigidity; gels; liquids; pastes; slurries; granules; powders; and other forms. The materials can be incorporated into special carriers such as liposomes or other vehicles to assist in their delivery either topically, gastrointestinally, intercavitary, or even intravascularly. In addition, combinations of these forms may also be used, for example, a bandage that combines a flexible, sponge-like or gel material that is placed directly onto a wound, and that has an outer protective backing of a somewhat rigid material that is easy to handle and manipulate, the outer layer providing mechanical protection to the wound after application. Both the inner and outer materials may contain clay minerals. Any means of administration may be used, so long as the mineral clay makes sufficient contact with the site of hemorrhage to promote hemostasis.

Compositions comprising clay minerals may be utilized to control bleeding in a large variety of settings, which include but are not limited to: (a) external bleeding from wounds (acute and chronic) through the use of liquids, slurries, gels, sprays, foams, hydrogels, powder, granules, or the coating of bandages with these preparations; (b) gastrointestinal bleeding through the use of an ingestible liquid, slurry, gel, foam, granules, or powder; (c) epistaxis through the use of an aerosolized powder, sprays, foam, patches, or coated tampon; (d) control of internal solid organ or boney injury through the use of liquids, slurries, sprays, powder, foams, gels, granules, or bandages coated with such; and (e) promotion of hemostasis, fluid absorption and inhibition of proteolytic enzymes to promote healing of all types of wound including the control of pain from such wounds.

Many applications of the present invention are based on the known problems of getting the surfaces of bandages to conform to all surfaces of a bleeding wound. The use of granules, powders, gels, foams, slurries, pastes, and liquids allow the preparations of the invention to cover all surfaces no matter how irregular they are. For example, a traumatic wound to the groin is very difficult to control by simple direct pressure or by the use of a simple flat bandage. However, treatment can be carried out by using a COMBINATION MATERIAL and sealant in the form of, for example, a powder, granule preparation, gel, foam, or very viscous liquid preparation that can be poured, squirted or pumped into the wound, followed by application of pressure. One advantage of the preparations of the present invention is their ability to be applied to irregularly shaped wounds, and for sealing wound tracks, i.e. the path of an injurious agent such as a bullet, knife blade, etc.

What is claimed is:

1. A method for promoting blood clotting comprising contacting a blood clot promoter with blood wherein said blood clot promoter comprises a COMBINATION Material comprising a mixture of a hydrophilic polymer and an inorganic material selected from the group consisting of diatomaceous earth, glass powder or fibers, precipitated or fumed silica, and calcium exchanged permutites.

2. The method of claim 1 wherein said hydrophilic polymer is selected from the group consisting of alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof.

3. The method of claim 1 wherein said hydrophilic polymer is chitosan.

4. The method of claim 1 wherein said blood clot promoter is contained within a porous carrier selected from the group consisting of woven fibrous articles, non-woven fibrous articles, puffs, sponges and mixtures thereof.

5. The method of claim 4 wherein said porous carrier is a woven or non-woven fibrous article comprising fibers selected from the group consisting of aramids, acrylics, cellulose, polyester, chemically modified cellulose fibers and mixtures thereof.

6. The method of claim 1 wherein the blood which is clotted comprises blood flowing from a wound in an animal or a human.

7. The method of claim 1 further comprising the step of removing all or a portion of said COMBINATION MATERIAL from a wound.

8. The method of claim 1 wherein said blood clot promoter is in the form of a free flowing powder.

9. The method of claim 1 wherein said blood clot promoter promotes blood clotting at a rate about 2-12 times faster than in its absence.

10. The method of claim 1 wherein said blood clot promoter promotes blood clotting in less than about 10 minutes.

11. The method of claim 1 wherein said blood clot promoter promotes blood clotting in less than about 5 minutes.

12. The method of claim 1 wherein said blood clot promoter further comprises antibiotics, antifungal agents, antimicrobial agents, anti-inflammatory agents, analgesics, bacteriostatics, compounds containing silver ions, fibrin(ogen), thrombin, superabsorbent polymers, calcium, polyethylene glycol, dextran, vasoactive catecholamines, vasoactive peptides, electrostatic agents, anesthetic agents or fluorescent agents.

13. The method of claim 1 wherein said blood clotting promoter is to treat blood hemorrhaging from an external wound.

14. The method of claim 1 wherein said blood clotting promoter is to treat blood hemorrhaging from an internal wound.

15. A blood clotting promoter comprising a mixture of a hydrophilic polymer and an inorganic material selected from the group consisting of diatomaceous earth, glass powder or fibers, precipitated or fumed silica, and calcium exchanged permutites.

16. The blood clotting promoter of claim 15 wherein said hydrophilic polymer is selected from the group consisting of alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof.

17. The blood clotting promoter of claim 15 wherein said hydrophilic polymer is chitosan.

18. The blood clotting promoter of claim 15 wherein said blood clot promoter is contained within a porous carrier selected from the group consisting of woven fibrous articles, non-woven fibrous articles, puff, sponges and mixtures thereof.

19. The blood clotting promoter of claim 18 wherein said porous carrier is a woven or non-woven fibrous article comprising fibers selected from the group consisting of aramids, acrylics, cellulose, polyester, chemically modified cellulose fibers and mixtures thereof.

20. The blood clotting promoter of claim 15 wherein said blood clot promoter is in the form of a free flowing powder.

21. The blood clotting promoter of claim 15 wherein said blood clot promoter further comprises antibiotics, antifungal agents, antimicrobial agents, anti-inflammatory agents, analgesics, bacteriostatics, compounds containing silver ions, fibrin(ogen), thrombin, superabsorbent polymers, calcium, polyethylene glycol, dextran, vasoactive catecholamines, vasoactive peptides, electrostatic agents, anesthetic agents or fluorescent agents.
